(19) United States
(12) Patent Application Publication
(10) Pub. No.: US 2007/0073231 A1
(43) Pub. Date: Mar. 29, 2007

(54) Medicinal solution dripping nozzle

(75) Inventors: Chang-Hyun Lee, Gyeonggi-do (KR); Tao-Seo Kim, Seoul (KR); Hyun-Ook Kim, Gyeonggi-do (KR); Jae-Hyun Park, Gyeonggi-do (KR)

Correspondence Address: BACON & THOMAS, PLLC, 625 SLATERS LANE, FOURTH FLOOR, ALEXANDRIA, VA, 22314, US

(73) Assignee: Hanmi Pharm. Co., Ltd., Kyungki-do (KR)

(21) Appl. No.: 11/434,878

(22) Filed: May 17, 2006

(30) Foreign Application Priority Data
Sep. 29, 2005 (KR) 10-2005-0091325

(51) Int. Cl. A61M 37/00

(52) U.S. Cl. 604/132

(57) ABSTRACT

A medicinal solution dripping nozzle is used to drop a predetermined dosage of medicinal solution in a form of droplet. The medicinal solution dripping nozzle includes a nozzle portion for defining a flow path, and a guide member fixedly positioned midway the nozzle portion. The guide member includes a base portion and a protrusion portion projected on the base portion wherein the base portion has at least two holes communicating with a medicinal solution chamber. Therefore, the medicinal solution is discharged through the holes from the medicinal solution chamber and guided to a tip end of the guide member while forming the droplet.

### FIELD OF THE INVENTION

The present invention relates to a medicinal solution dripping nozzle for dripping a predetermined dosage of medicinal solution in a form of droplet, and in particular to a medicinal solution dripping nozzle capable of evenly dispensing a desired dosage of medicinal solution and a medicinal solution container having the medicinal solution dripping nozzle.

### BACKGROUND OF THE INVENTION

In order to cure or prevent a disease or injury for eyes, it is often required to drop eye drop solution, saline solution, or the like to an eye ball, an injured area, or the like in a form of droplet. As such, there has been developed a number of medicinal solution containers such as a medicinal solution chamber. When the medicinal solution chamber is manually compressed with a small pressure, the medicinal solution contained in the chamber is discharged through a nozzle in a form of droplet. Such chambers are commercially available and ease to purchase in markets.

A conventional nozzle used in a medicinal solution chamber takes a form of a hollow conical nozzle or a hollow cylindrical nozzle, in which medicinal solution is guided to a tip end of the nozzle through a hollow portion thereof and then discharged in a form of droplet from the tip end of the nozzle.

With such a conventional nozzle, the size of a droplet discharged from the nozzle is caused to vary depending on the intensity of pressure manually applied to the medicinal solution chamber, the length of time for applying the pressure, the tilting angle of the nozzle, or the like, and medicinal solution of about 24 mg/droplet to 26 mg/droplet is dispensed regardless the type of the medicinal solution.

If there is no need to make the discharge dosage of medicinal solution even, there is no problem in using such a conventional nozzle. However, if it is needed for the discharge dosage of medicinal solution to be even, such a conventional nozzle cause a problem of wrong or extravagant use of medicinal solution.

In particular, the dosage of an eye drop solution containing, for example, Prostamide component for use in curing glaucoma is extremely limited to one droplet per day, which corresponds to about 31 mg. If the Prostamide component is applied to an eyeball in excess of the prescribed dosage, the coloring tissue of the eyeball is caused to change, whereby there may be an adverse reaction which causes coloring matter to be deposited to the iris or changes the color of the eye.

In addition, because such an adverse reaction is very slowly progressed, the patient may not be conscious of the adverse reaction until the lapse of several months or several years. Accordingly, there is a problem in that it is impossible to use the medicinal solution chamber with the above-mentioned conventional nozzle in applying some kinds of medicinal solution.

Furthermore, if a nozzle has only one hole for discharging medicinal solution, air bubbles are apt to be formed in the medicinal solution in a process of forming a droplet of the medicinal solution, and it is difficult to reintroduce extra medicinal solution, which remains in the nozzle after the formation and discharge of the droplet, into the medicinal solution chamber, which may cause the waste or degeneration of the medicinal solution.

In particular, if the size of the hole is increased so as to increase the size of the droplet of the medicinal solution, thereby increasing the discharge dosage of medicinal solution, there may occur a problem in that the deviation in discharge dosage of medicinal solution is increased depending on the increase of the size of the hole, whereby it is impossible to assure the even discharge of the medicinal solution, and the medicinal solution flows out spontaneously through the hole even though the medicinal solution container is tilted.

### SUMMARY OF THE INVENTION

Therefore, it is an object of the present invention to provide a medicinal solution dripping nozzle capable of evenly dispensing a desired dosage of medicinal solution and capable of easily reintroducing extra medicinal solution into the medicinal solution chamber.

Another object of the present invention is to provide a medicinal solution container having the medicinal solution dripping nozzle.

In accordance with one aspect of the present invention, there is provided a medicinal solution dripping nozzle for use in a medicinal solution chamber to drop a predetermined dosage of medicinal solution in a form of droplet, which comprises: a nozzle portion for defining a flow path; and a guide member fixedly positioned midway the nozzle portion, the guide member including a base portion and a protrusion portion projected on the base portion and the base portion having a plurality of holes communicating with the medicinal solution chamber, whereby the medicinal solution is discharged through the holes from the medicinal solution chamber and guided to a tip end of the guide member protrusion portion along an inner wall of the nozzle portion through the holes while forming the droplet.

In accordance with another aspect of the present invention, there is provided a medicinal solution container, comprising: a medicinal solution chamber containing a medicinal solution therein; and a nozzle for dripping a droplet of a predetermined dosage of the medicinal solution in the medicinal solution chamber to a predetermined area,

wherein the dripping nozzle includes: a nozzle portion for defining a flow path to pass the medicinal solution therethrough; a guide member fixedly positioned midway the nozzle portion, the guide member including a base portion and a protrusion portion projected on the base portion, and the base portion having a plurality of holes communicating with the medicinal solution chamber; and a connecting portion for coupling the medicinal solution chamber and the nozzle, whereby the medicinal solution is discharged through the holes from the medicinal solution chamber and guided to a tip end of the guide member while forming the droplet.

### BRIEF DESCRIPTION OF THE DRAWINGS

The above and other objects, features and advantages of the present invention will be more apparent from the following detailed description taken in conjunction with the accompanying drawings, in which:

FIG. 1 is an exploded perspective view of a medicinal solution container with a medicinal solution dripping nozzle according to a preferred embodiment of the present invention;

FIG. 2A shows a cross-sectional view of the medicinal solution dripping nozzle taken along line A-A of FIG. 1;

FIG. 2B shows a partially enlarged view of the medicinal solution dripping nozzle of FIG. 1;

FIG. 2C shows a top plan view of the medicinal solution dripping nozzle of FIG. 1;

FIGS. 3A to 3C show a modified example for the guide member of FIG. 1, which correspond to the cross-sectional view, the partially enlarged view and the top plan view of FIGS. 2A to 2C, respectively;

FIGS. 4A to 4C show another modified example for the guide member of FIG. 1, which correspond to the cross-sectional view, the partially enlarged view and the top plan view of FIGS. 2A to 2C, respectively;

FIGS. 5 and 6 show schematic sequential diagrams illustrating formation of droplet of solution in sequence;

FIG. 7 is an exploded perspective view of a medicinal solution container with a medicinal solution dripping nozzle according to another embodiment of the present invention;

FIG. 8A shows a cross-sectional view taken along line B-B of FIG. 7;

FIG. 8B shows a partially enlarged view of the medicinal solution dripping nozzle of FIG. 7; and

FIG. 8C shows a top plan view of the medicinal solution dripping nozzle of FIG. 7.

### DETAILED DESCRIPTION OF THE PREFERRED EMBODIMENTS

Hereinafter, preferred embodiments of the present invention will be described in detail with reference to accompanying drawings, wherein same or corresponding components in overall drawings will be designated like reference numerals.

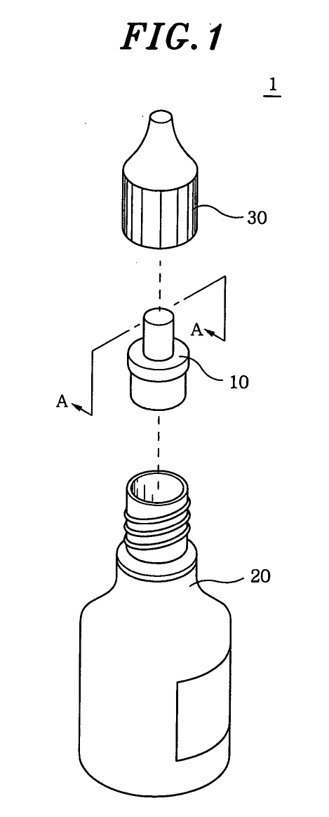

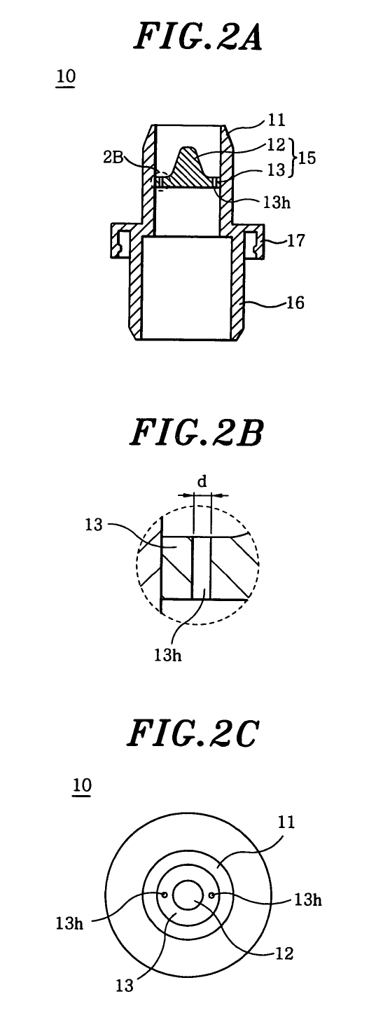

Referring to FIG. 1, there is shown an exploded perspective view of a medicinal solution container with a medicinal solution dripping nozzle according to a preferred embodiment of the present invention. Further, FIG. 2A shows a cross-sectional view of the medicinal solution dripping nozzle taken along line A-A of FIG. 1; FIG. 2B shows a partially enlarged view of the medicinal solution dripping nozzle of FIG. 1; and FIG. 2C shows a top plan view of the medicinal solution dripping nozzle of FIG. 1.

As shown in FIG. 1, a medicinal solution container comprises a medicinal solution dripping nozzle 10, a medicinal solution chamber 20 for containing medicinal solution therein, and a lid 30 for enclosing the medicinal solution dripping nozzle 10.

The medicinal solution dripping nozzle 10, as shown in FIGS. 2A to 2C, includes a nozzle portion 11 formed by a cylindrical tube to define a flow path, through which the medicinal solution is dispensed from the medicinal solution chamber 20; a guide member 15 fixedly positioned halfway the nozzle portion 11; and a connecting portion 16 for interconnecting the nozzle portion 11 and the medicinal solution chamber 20 along with a hooking part 17.

The guide member 15 is made from a flexible material and is formed with a base portion 13 and a protrusion portion 12 projected from a center of the base portion 13. The base portion 13 has one or more holes 13h along its periphery, which communicate with the interior of the medicinal solution chamber 20.

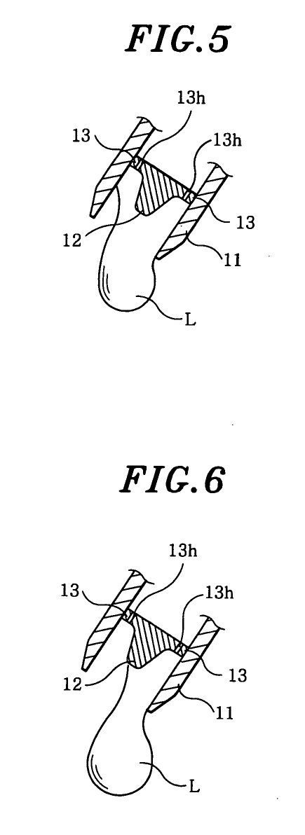

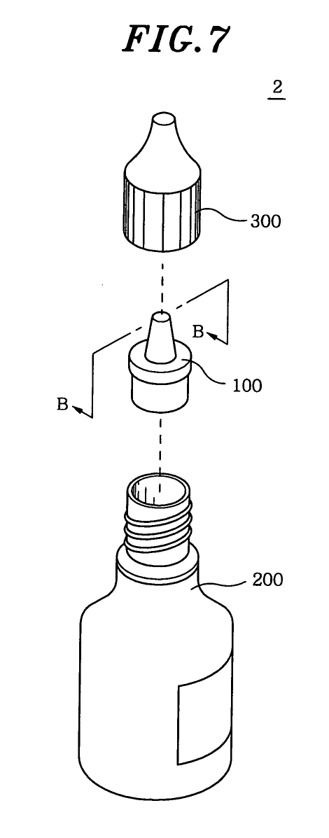

When the medicinal solution chamber 20 is compressed by a hand (not shown) with a small pressure or the like, the medicinal solution is discharged through the holes 13h from the medicinal solution chamber 20 and guided to an end of the nozzle portion 11, as shown in FIG. 5. Then, the solution is retained in a form of droplet at the tip ends of an inner wall of the nozzle portion 11 and the protrusion portion 12 by the surface tension of the medicinal solution as shown in FIG. 6. Subsequently, the droplet is released from the tip ends of the inner wall of the nozzle portion 11 and the protrusion portion 12 and dropped to a predetermined area.

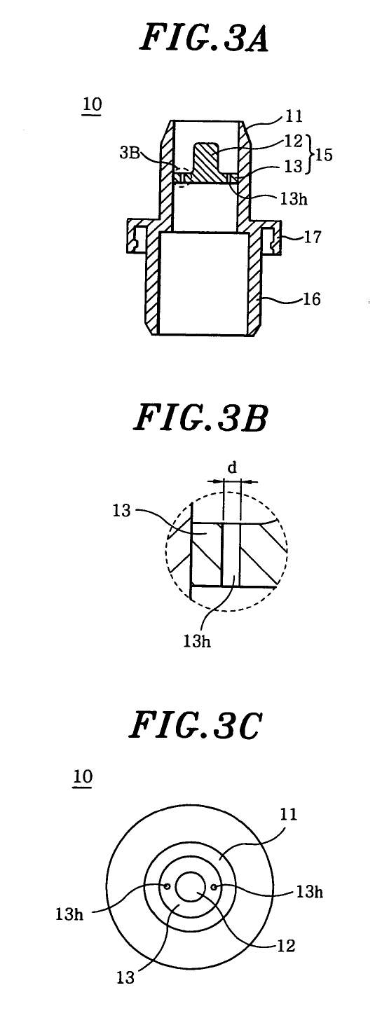

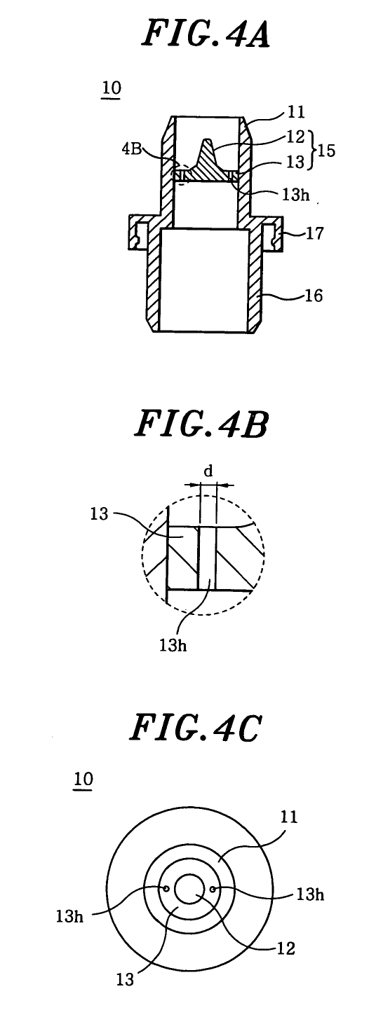

In order to facilitate the formation of the droplet, the guide portion 12 may be formed in a variety of shapes, e.g., a truncated conical shape, a cylindrical shape, a conical shape, or the like as shown in FIGS. 2A to 4C, respectively. In addition, it is preferred that the guide member 15 has at least two holes and the holes are arranged to be symmetrical to each other, wherein the diameter of each hole is larger than 0.15 mm and smaller than 0.25 mm.

As the diameter of each hole 13h is increased, the discharge dosage of medicinal solution is increased while the standard deviation in discharge dosage is increased. Whereas, as the diameter of the holes 13h is reduced, the standard deviation in discharge dosage is reduced while the discharge dosage is reduced.

Therefore, it is required to increase the number of the holes 13h in order to reduce the standard deviation in discharge dosage while simultaneously increasing the discharge dosage of medicinal solution through the holes 13h.

In order to confirm the above requirement, experimental Examples 1 to 6 and Comparative Examples 1 to 3 were conducted as follows.

For the purpose of discharging an eye drop solution of, e.g., Prostamide component, in a dosage of 31 mg/droplet at the atmospheric pressure (1 atm) and the normal temperature (25° C.), experiments for discharging one droplet were conducted using a nozzle having one hole (Example 1) and using a nozzle 10 having two holes while changing the size of the holes (Examples 2 to 4), the results of which are listed in Table 1 below.

TABLE 1

| | Example 1 | Example 2 | Example 3 | Example 4 |
|---|---|---|---|---|
| Number of holes | 1 | 2 | 2 | 2 |
| Diameter of hole | 0.4 | 0.1 | 0.2 | 0.3 |
| (mm) | | | | |
| Average (mg) | 31.8 | 30 | 31.7 | 32.1 |
| Standard deviation | 2.7 | 2.1 | 1.5 | 3.5 |
| (mg) | | | | |
| Maximum value (mg) | 37 | 35.1 | 35.8 | 36.9 |
| Minimum value (mg) | 26.6 | 24.5 | 27.8 | 21.4 |
| Maxim value - | 10.4 | 10.6 | 8 | 15.5 |
| Minimum value (mg) | | | | |
| Number of times of | 60 | 60 | 160 | 80 |
| discharge (times) | | | | |

As indicated on the Table 1 comparing the increase of the size of holes and the number of the holes in order to increase the discharge dosage of medicinal solution, it is observed that the standard deviation in discharge dosage of medicinal solution becomes high in case where the size of the holes is large as in Example 1. Therefore, it is recommended to increase the number of holes more than one in order to obtain an even discharge dosage of medicinal solution while approximately satisfying a target discharge dosage (31 mg).

In addition, in Examples 2 to 4, in case that the size of each hole is 0.1 mm, it is needed for a user to compress the medicinal solution chamber 20 for a long period of time because the amount of discharging a desired dosage of medicinal solution through the small-sized hole is small, and in case that the diameter of each hole 13h is 0.3 mm, the standard deviation in discharge dosage of medicinal solution becomes high and the medicinal solution flows out spontaneously even when the medicinal solution container 20 is tilted.

Moreover, upon comparing Example 2 and Example 4 with Example 3, it was observed that the difference between the maximum value and minimum value in discharge dosage was substantially reduced in Example 3.

For the purpose of discharging an eye drop solution of Prostamide component in a dosage of 31 mg/droplet at the atmospheric pressure (1 atm) and the normal temperature (25° C.), experiments for discharging one droplet were conducted using a nozzle 10 having two holes while changing the diameter of the holes (Examples 5 and 6), the results of which are listed in Table 2 below.

TABLE 2

| | Example 5 | Example 3 | Example 6 |
|---|---|---|---|
| Number of holes | 2 | 2 | 2 |
| Diameter of hole (mm) | 0.15 | 0.2 | 0.25 |
| Average (mg) | 30.2 | 31.7 | 32.6 |
| Standard deviation (mg) | 2.2 | 1.5 | 2.6 |
| Maximum value (mg) | 33.4 | 35.8 | 38 |
| Minimum value (mg) | 24.9 | 27.8 | 29.6 |
| Maxim value - Minimum value | 8.5 | 8 | 8.4 |
| (mg) | | | |
| Number of times of discharge | 30 | 160 | 30 |
| (times) | | | |

According to these experiments, it was observed that the difference between the maximum value and minimum value in discharge dosage of medicinal solution was also reduced even in case that the diameters of holes are 0.15 mm and 0.25 mm, respectively. Therefore, it can be appreciated that the diameter of each hole can be selected within a predetermined range, i.e., 0.15 mm to 0.25 mm, including 0.2 mm.

Additionally, experiments for Comparative Examples 1 to 3 for discharging one droplet of eye drop solution of Prostamide component were conducted at the atmospheric pressure (1 atm) and the normal temperature (25° C.) using a conventional nozzle while tilting the nozzle, the results of which are listed in Table 3 below.

TABLE 3

| | Comparative | Comparative | Comparative |
|---|---|---|---|
| | Example 1 | Example 2 | Example 3 |
| Number of holes | 1 | 1 | 1 |
| Diameter of hole | 0.2 | 0.2 | 0.2 |
| (mm) | | | |
| Average (mg) | 26.1 | 44.8 | 32.8 |
| Standard | 3.1 | 2.1 | 8 |
| deviation (mg) | | | |
| Maximum value | 36.9 | 52.2 | 45.7 |
| (mg) | | | |
| Minimum value | 21.4 | 40.2 | 22.2 |
| (mg) | | | |
| Maxim value - Minimum | 15.5 | 12 | 23.5 |
| value | | | |
| (mg) | | | |
| Number of times | 30 | 30 | 30 |
| of discharge | | | |
| (times) | | | |

According to the experiments for Comparative Examples, it was observed that, in case where one hole is formed in the guide member, the standard deviation in discharge dosage of medicinal solution and the difference between the maximum value and minimum value of the discharge dosage are increased depending on the tilting angle of the nozzle, and the standard deviation and the difference are also high even if the tilting angle remains constant.

In addition, experiments were conducted to discharge one droplet of an eye drop solution of Prostamide component under the condition of the atmospheric pressure (1 atm) and the normal temperature (25° C.) using a conventional nozzle and the inventive nozzle with two holes, each having a diameter of 0.2 mm, while tilting the nozzles to 0, 23, 45, 68 and 90 degrees with respect to the horizontal, the results of which are listed in Table 4 below. Further, the number of times of discharging medicinal solution was set to 20 for each tilting angle.

TABLE 4

| | | Conventional | | | |
|---|---|---|---|---|---|
| | | Nozzle | | Inventive nozzle | |
| | | | Standard | | Standard |
| | | Average | deviation | Average | deviation |
| Tilting | 0 Deg. | 19 | 3.9 | 24.3 | 0.8 |
| angle from | 23 Deg. | 23.1 | 2.7 | 26.2 | 0.7 |
| horizontal | 45 Deg. | 25 | 1.1 | 31.9 | 0.7 |
| | 68 Deg. | 25.9 | 0.8 | 32.6 | 1.1 |
| | 90 Deg. | 28.1 | 0.7 | 34.4 | 1.1 |
| | Average | 24.2 | 1.8 | 30 | 0.9 |

According to these experiments, it was observed that, in the conventional nozzle, the discharge dosage of medicinal solution and the standard deviation in the discharge dosage are greatly changed depending on the tilting angles of the medicinal solution container at the time of discharging the medicinal solution from, whereas it was observed that, in case of the inventive nozzle having two holes, each having a diameter of 0.2 mm, the discharge dosage of medicinal solution and the standard deviation in discharge dosage were greatly reduced as compared to the conventional nozzle even if the medicinal solution container 1 is equally tilted.

Therefore, it can be appreciated that the nozzle with two holes, each having a diameter of 0.2 mm, is more advantageous so as to keep the discharge dosage even, regardless of the tilting angle of the medicinal solution container 1.

Accordingly, in order to assure a desired discharge dosage of medicinal solution to be kept even, it is preferable that the inventive medicinal solution dripping nozzle has two or more holes for discharging medicinal, the diameter of each hole is larger than 0.1 mm and smaller than 0.3 mm, more preferably, larger than 0.15 mm and smaller than 0.25 mm.

Needless to say, the discharge dosage of medicinal solution can be increased by increasing the number of holes.

In particular, in case of the eye drop solution of Prostamide component that is a strong and expensive medicine for curing glaucoma, it is necessary to apply it to an eyeball one time per day in a dosage of 31 mg. Therefore, in order to meet the requirement, it is preferred that the nozzle has two holes for discharging medicinal solution and the diameter of each hole is 0.2 mm, as can be seen from the results of Examples 1 to 6.

Furthermore, the nozzle having two or more holes 13h may further reduce the occurrence of air bubbles in the course of discharging medicinal solution than having only one hole because the area, over which the medicinal solution is needed to be spread, is reduced.

More specifically, in order to reduce the occurrence of air bubbles in case of two holes formed in the nozzle, it is desired for the two holes to be remotely spaced from each other as much as possible so that the solution is spread over a wide range of region near to the hole 13h. Therefore, it is preferable to form the two holes 13h in such a manner as to be symmetrically arranged with reference to the guide portion 12.

As the holes are formed passing through the base portion 13, the extra medicinal solution remaining on the tip end of the guide member 15 after discharging a droplet of the medicinal solution, can be easily reintroduced into and received in the medicinal solution chamber 20.

In addition, as shown in FIGS. 2A to 6, the tip end of the protrusion portion 12 is rounded so as to minimize the surface tension of a droplet at the tip end of the protrusion portion 12, whereby the droplet can be easily released from the rounded tip end of the protrusion portion 12.

Although the present invention has been described with respect to the cases in which the guide member 15 of the medicinal solution dripping nozzle 10 is formed by a cylindrical tube, the guide member of the medicinal solution dripping nozzle may be formed in a hollow and truncated conical shape.

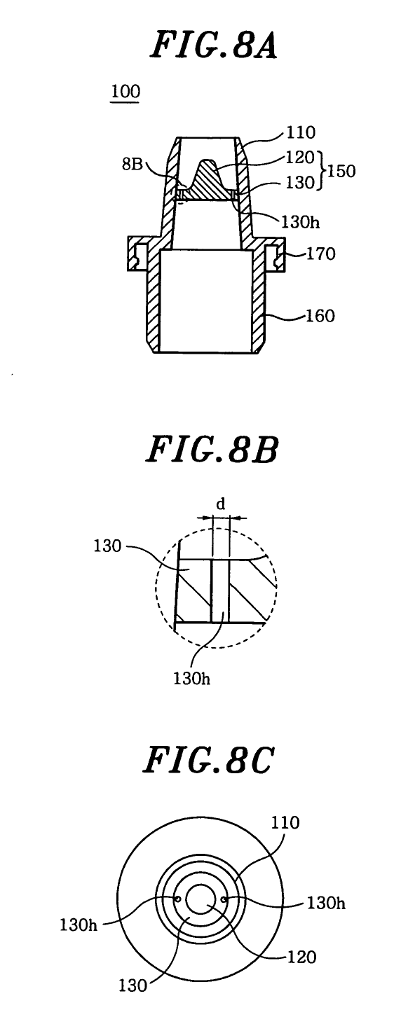

FIG. 7 is an exploded perspective view of a second embodiment of a medicinal solution container with a medicinal solution dripping nozzle. Further, FIG. 8A shows a cross-sectional view taken along line B-B of FIG. 7; FIG. 8B shows a partially enlarged view of the medicinal solution dripping nozzle of FIG. 7; and FIG. 8C shows a top plan view of the medicinal solution dripping nozzle of FIG. 7.

The components of the second embodiment are substantially identical to those of the first embodiment, except that the medicinal solution dripping nozzles 1 and 2 are different from each other in their shapes, and therefore, a detailed description thereof will be omitted for the sake of simplicity.

More specifically, the inventive medicinal solution dripping nozzle 100 includes a nozzle portion 110 formed in a truncated conical shape to define a flow path, through which the medicinal solution is dispensed from the medicinal solution chamber 200; a guide member 150 fixedly positioned halfway the nozzle portion 110; and a connecting portion 160 for interconnecting the nozzle portion 110 and the medicinal solution chamber 200 along with a hooking part 170.

The guide member 150 is made from a flexible material and is formed with a base portion 130 and a protrusion portion 120 projected from a center of the base portion 130. The base portion 130 has one or more holes 130h along its periphery, which communicate with the interior of the medicinal solution chamber 200.

Similarly, as shown in FIGS. 5 and 6, if the medicinal solution chamber 200 is compressed, the medicinal solution discharged through the holes 130h is guided to the tip ends of the nozzle portion 110 and the guide member 150, retained therebetween by surface tension of the medicinal solution, thereby forming a droplet L, and then released from the tip end, thereby being dropped to a desired area.

Although not shown in the accompanying drawings, if the pressure applied to the medicinal solution chamber 200 is reduced after the droplet L is dropped, the ambient air is introduced into the medicinal solution chamber through the holes 130h, thereby causing the medicinal solution remaining on the base portion 130 to be introduced into the medicinal solution chamber 200.

As described above, according to the present invention, there is provided a medicinal solution dripping nozzle, which reduces the standard deviation in discharge dosage of medicinal solution discharged from a medicinal solution chamber, allows the discharge dosage of medicinal solution to be controlled, and causes the medicinal solution, which remains in the nozzle after a droplet of the medicinal solution is dropped, to be easily reintroduced into the medicinal solution chamber.

In particular, if the inventive medicinal solution dripping nozzle is used in combination with a container for an eye drop solution, which should be discharged in a precise dose, it is possible to apply a precise dose of the eye drop solution to an eyeball, whereby an adverse reaction, which may be caused from wrong or extravagant use of medicinal solution, can be prevented.

While the invention has been shown and described with respect to the preferred embodiments, it will be understood by those skilled in the art that various changes and modifications may be made without departing from the spirit and scope of the invention as defined in the following claims.

What is claimed is:

1. A medicinal solution dripping nozzle for use in a medicinal solution chamber to drop a predetermined dosage of medicinal solution in a form of droplet, comprising:
a nozzle portion for defining a flow path; and
a guide member fixedly positioned midway the nozzle portion, the guide member including a base portion and a protrusion portion projected on the base portion and the base portion having a plurality of holes communicating with the medicinal solution chamber, whereby the medicinal solution is discharged through the holes from the medicinal solution chamber and guided to a tip end of the guide member while forming the droplet.

2. The medicinal solution dripping nozzle of claim 1, wherein the base portion has two holes formed along its periphery and arranged to be symmetrical to each other.

3. The medicinal solution dripping nozzle of claim 1, wherein the diameter of each hole is larger than 0.1 mm and smaller than 0.3 mm, and preferably is larger than 0.15 mm and smaller than 0.25 mm.

4. The medicinal solution dripping nozzle of claim 1, wherein the nozzle portion is formed in a cylindrical shape.

5. The medicinal solution dripping nozzle of claim 1, wherein the nozzle portion is formed in a truncated shape.

6. The medicinal solution dripping nozzle of claim 1, wherein the protrusion portion has a cylindrical shape, and the tip end of which is rounded.

7. The medicinal solution dripping nozzle of claim 1, wherein the protrusion portion has a conical shape, and the tip end of which is rounded.

8. The medicinal solution dripping nozzle of claim 1, wherein the medicinal solution is an eye drop solution.

9. A medicinal solution container comprising:
a medicinal solution chamber containing a medicinal solution therein;
a nozzle for dripping a droplet of a predetermined dosage of the medicinal solution in the medicinal solution chamber to a predetermined area,
wherein the dripping nozzle includes:
a nozzle portion for defining a flow path to pass the medicinal solution therethrough;
a guide member fixedly positioned midway the nozzle portion, the guide member including a base portion and a protrusion portion projected on the base portion, and the base portion having a plurality of holes communicating with the medicinal solution chamber; and
a connecting portion for coupling the medicinal solution chamber and the nozzle, whereby the medicinal solution is discharged through the holes from the medicinal solution chamber and guided to a tip end of the guide member while forming the droplet.

10. The medicinal solution container of claim 9, wherein the diameter of each hole is larger than 0.1 mm and smaller than 0.3 mm, and preferably, is larger than 0.15 mm and smaller than 0.25 mm.

11. The medicinal solution container of claim 9, wherein the base portion includes at least two holes formed along its periphery and arranged to be symmetrical to each other.

12. The medicinal solution container of claim 9, wherein the protrusion portion has a cylindrical shape, the tip end of which is rounded.

13. The medicinal solution container of claim 9, wherein the protrusion portion has a conical shape, the tip end of which is rounded.

14. The medicinal solution container of claim 9, wherein the medicinal solution is an eye drop solution.
